SEQUENCE LISTING (4 sequences)

SEQ ID NO: 1 - protein, 1273 aa, Artificial sequence
  surface spike glycoprotein of SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 223 aa, Artificial sequence
  amino acids R319-F541 of the receptor binding domain (RBD) of surface spike glycoprotein of SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 3 - protein, 518 aa, Artificial sequence
  Amino acids M697-P1213 of the S2 region of the surface spike glycoprotein of SARS-CoV-2
MSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPF

SEQ ID NO: 4 - protein, 419 aa, Artificial sequence
  Amino acids M1-A419 of the SARS-CoV-2 nucleocapsid protein.
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

(19) United States
(12) Patent Application Publication
(10) Pub. No.: US 2022/0120737 A1
(43) Pub. Date: Apr. 21, 2022

(54) METHOD FOR DETECTING SARS-COV-2-SPECIFIC SERUM HUMAN IMMUNOGLOBULINS

(71) Applicant: RayBiotech Life, Peachtree Corners, GA (US)

(72) Inventor: Ruo-Pan Huang, Johns Creek, GA (US)

(21) Appl. No.: 17/076,034

(22) Filed: Oct. 21, 2020

(51) Int. Cl. G01N 33/543; G01N 33/68

(52) U.S. Cl. CPC G01N 33/54306; G01N 2333/165; G01N 33/6854

(57) ABSTRACT

A high-throughput high sensitivity and high selectivity ELISA-based method is provided that can detect human IgA, IgM, and IgG directed against SARS-CoV-2 with minimum false positive results. Simultaneous use of SARS-CoV-2-specific antigens derived from SARS-CoV-2 spike and nucleocapsid proteins provides greater sensitivity and selectivity compared to current methods for the detection of SARS-CoV-2-induced human immunoglobulin A, M, and G serum antibodies.

### SEQUENCE LISTING

This application contains a sequence listing filed in electronic form as an ASCII.txt file entitled “2208031100_ST25” created on Oct. 5, 2020. The content of the sequence listing is incorporated herein in its entirety.

### TECHNICAL FIELD

The present disclosure is generally related to methods of detecting human antibodies specifically binding SARS-CoV-2 nucleocapsid and spike protein antigens. The present disclosure is also generally related to kits for the performance of the methods of the disclosure.

### BACKGROUND

The world is challenged by the pandemic caused by a new coronavirus, SARS-CoV-2. SARS-CoV-2 infects humans and can cause serious pneumonia complicated by extensive inflammatory cytokines production (a “cytokine storm”). The virus-associated disease has typical symptoms of pneumonia accompanied by several other symptoms e.g. muscle pain, headache and sore throat and is most commonly referred to as COVID-19.

Currently there are no known effective therapeutic treatments for a COVID-19 infection. Therefore, the prognosis largely depends on the efficacy of the host's immune system. Early diagnostic kits directed against virus-specific IgM and IgG antibodies, or q-RT-PCR kits detecting viral levels have been developed, but the supply has been in a great shortage in the United States. In addition, the high percentage of false positive results, mainly caused by high levels of immunoglobulin in certain patients, has also been a challenge for serum anti-virus immunoglobulin (Ig) detection.

### SUMMARY

Embodiments of a multiplex bead-based high-throughput high sensitivity diagnosis method that can detect human IgG, IgA and IgM directed against SARS-CoV-2 simultaneously, with minimum false positive results is provided. Instead of comparing the absolute read signal, this kit introduces an internal control as background reference for each specific sample. By comparing the ratio of signals between viral antigen-coated beads and control protein-coated beads the real signal due to anti-viral Ig can be determined.

One aspect of the disclosure encompasses embodiments of a method of detecting an immune response to SARS-CoV-2, the method comprising: (a) incubating, under conditions effective to allow immune complex formation, a serum sample from a subject suspected of having been exposed to a SARS-CoV-2 virus with a mixture of a SARS-CoV-2 spike protein, or fragments thereof, and a SARS-CoV-2 nucleocapsid protein, or fragments thereof, wherein the mixture of SARS-CoV-2 proteins or fragments thereof are bound to a plurality of wells of a multi-well plate; (b) incubating, under conditions effective to allow immune complex formation, the serum sample from the subject with a serum albumin, wherein the serum albumin is bound to a control well of a multi-well plate; (c) washing unbound serum samples from the wells; (d) adding a biotinylated anti-human IgA antibody, a biotinylated anti-human IgM, or a biotinylated antibody anti-human IgG antibody, or biotinylated antigen-binding fragments thereof, to the well having the bound SARS-CoV-2 proteins or fragments thereof, and to the control well; (e) incubating the wells for a period effective to allow the anti-human Ig antibody, or antigen-binding fragments thereof, to bind to serum anti-SARS-CoV-2 antibodies bound to the SARS-CoV-2 proteins or fragments thereof, and washing the wells to remove unbound biotinylated anti-human Ig antibody; (f) adding a horse radish peroxidase (HRP)-streptavidin conjugate and an HRP substrate to each of the wells from step (e), thereby generating a light detectable product; (g) determining the light absorbance of the product from step (f) for each of the wells; (h) subtracting the absorbance measured for the control well from the absorbance of the well having the bound SARS-CoV-2 proteins or fragments thereof; and (i) calculating the amount of Ig in the serum sample from the measured absorbance of the well having the bound SARS-CoV-2 proteins or fragments thereof minus the measured absorbance of the control well.

Another aspect of the disclosure encompasses embodiments of a kit comprising: (i) a vessel or vessels containing at least one of a biotinylated anti-human IgA antibody, a biotinylated anti-human IgM antibody, and a biotinylated anti-human IgG antibody, or antigen-binding fragments thereof; (ii) at least one multi-well plate comprising a plurality of wells, wherein the wells are coated with a mixture of a SARS-CoV-2 spike protein, or a fragment thereof, and a SARS-CoV-2 nucleocapsid protein, or fragments thereof; (iii) a vessel containing a horse radish peroxidase-streptavidin conjugate; and (iv) instructions for the use of the reagents of the kit in the method of claim 1 for the detection of at least one of aSARS-CoV-2-specific IgA, IgM or IgG antibody.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates the assay of the disclosure.

FIG. 2 illustrates a table showing a comparison of performance of different methods.

FIG. 3 illustrates a table showing a comparison of performance of different methods.

FIG. 4 illustrates a pair of graphs illustrating the performance of Sars-Cov-2-SRBD IgG and SRBD+N IgG.

FIG. 5 illustrates a pair of graphs illustrating the performance of Sars-Cov-2-SRBD IgG and SRBD+N IgG.

FIG. 6 illustrates a pair of graphs illustrating the performance of Sars-Cov-2-SRBD IgG and SRBD+N IgM.

FIG. 7 illustrates a pair of graphs illustrating the performance of Sars-Cov-2-SRBD IgG and SRBD+N IgM.

FIG. 8 illustrates a pair of graphs illustrating the performance of Sars-Cov-2-SRBD IgG and SRBD+N IgA.

FIG. 9 illustrates a pair of graphs illustrating the performance of Sars-Cov-2-SRBD IgG and SRBD+N IgA.

FIG. 10 illustrates a pair of graphs illustrating the correlation between serum and plasma.

FIG. 11 illustrates a pair of graphs illustrating the correlation between serum and DBS.

FIG. 12 illustrates the amino acid sequence of the surface spike glycoprotein of SARS-CoV-2 (SEQ ID NO: 1), amino acids R319-F541 of the receptor binding domain (RBD) (SEQ ID NO: 2), and amino acids M697-P1213 (SEQ ID NO: 3) of the S2 region of SEQ ID NO: 1.

FIG. 13 illustrates the amino acid sequence M1-A419 of the SARS-CoV-2 (SEQ ID NO: 4), of the SARS-CoV-2-derived nucleocapsid protein.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Abbreviations

ELISA, Enzyme Linked Immunoglobulin Sandwich Assay; BSA, Bovine Serum Albumin; TMB, 3,3,5,5′-tetramethylbenzidine; SARS-CoV-2, Severe Acute Respiratory Syndrome Coronavirus 2; COVID-19, coronavirus disease 2019; RBD receptor binding domain;

### Definitions

The term “specific binding” as used herein refers to the specific recognition of one molecule, of two different molecules, compared to substantially less recognition of other molecules. Generally, the molecules have areas on their surfaces or in cavities giving rise to specific recognition between the two molecules. Exemplary of specific binding are antibody-antigen interactions.

The term “antibody” as used herein refers to an immunoglobulin which specifically binds to and is thereby defined as complementary with a particular spatial and polar organization of another molecule. The antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, IgY, etc. Fragments thereof may include Fab, Fv and F(ab′)2, Fab′, scFv, and the like. In addition, aggregates, polymers, and conjugates of immunoglobulins or their fragments can be used where appropriate so long as binding affinity for a particular molecule is maintained.

Antibodies may be derived from any source, including, but not limited to, murine spp., rat, rabbit, chicken, human, or any other origin (including humanized antibodies). Techniques for the generation of antibodies that can specifically recognize and bind to are known in the art.

The term “antigen” as used herein refers to any entity that binds to an antibody and induces at least one shared conformational epitope on the antibody. Antigens can be proteins, peptides, antibodies, small molecules, lipid, carbohydrates, nucleic acid, and allergens. An antigen may be in its pure form or in a sample in which the antigen is mixed with other components. In particular, the methods of the present disclosure are intended to detect human or animal immunoglobulins that specifically recognize and bind to epitopes of the S and/or N polypeptides of the SARS-CoV-2 virus.

The term “Severe Acute Respiratory Syndrome Coronavirus 2 (SARS-CoV-2)” as used herein refers to is the strain of coronavirus that causes coronavirus disease 2019 (COVID-19), the respiratory illness responsible for the COVID-19 pandemic. Colloquially known as simply the coronavirus, it was previously referred to by its provisional name, 2019 novel coronavirus (2019-nCoV), and has also been called human coronavirus 2019 (HCoV-19 or hCoV-19). SARS-CoV-2 is a Baltimore class IV positive-sense single-stranded RNA virus that is contagious in humans. It is the successor to SARS-CoV-1, the strain that caused the 2002-2004 SARS outbreak.

Each SARS-CoV-2 virion is 50-200 nm in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. The spike protein, which has been imaged at the atomic level is responsible for allowing the virus to attach to and fuse with the membrane of a host cell; specifically, its 51 subunit catalyzes attachment, the S2 subunit fusion.

SARS-CoV-2 has sufficient affinity to the receptor angiotensin converting enzyme 2 (ACE2) on human cells to use them as a mechanism of cell entry. Studies have shown that SARS-CoV-2 has a higher affinity to human ACE2 than the original SARS virus strain.

Initial spike protein priming by transmembrane protease, serine 2 (TMPRSS2) is essential for entry of SARS-CoV-2. After a SARS-CoV-2 virion attaches to a target cell, the cell's protease TMPRSS2 cuts open the spike protein of the virus, exposing a fusion peptide in the S2 subunit, and the host receptor ACE2. After fusion, an endosome forms around the virion, separating it from the rest of the host cell. The virion escapes when the pH of the endosome drops or when cathepsin, a host cysteine protease, cleaves it. The virion then releases RNA into the cell and forces the cell to produce and disseminate copies of the virus, which infect more cells.

### Discussion

The methods of the disclosure encompass the use of a combination of SARS-CoV-2-specific antigens, and in particular a combination of SARS-CoV-2 nucleocapsid (N) protein-specific-antigens and Spike protein-specific antigens. It has been surprisingly found that this combination significantly increases the specificity and the sensitivity of a sandwich-based ELISA assay method configured to detect the presence of human serum antibodies specific binding the two SARS-CoV-2 proteins. A further improvement in sensitivity and selectivity is provided by subtracting from the determinations non-specific binding of the anti-human immunoglobulin antibodies by using an albumin control. Accordingly, the ELISA-based assays of the disclosure can be configured for the detection of immunoglobulin (Ig) A, and G (respectively, IgA, IgM, and IgG) either alone or simultaneously.

In an exemplary “sandwich” ELISA of the disclosure, at least one polypeptide comprising antigens of the SARS-CoV-2 (CoViD19) may be immobilized onto a selected surface exhibiting protein affinity, such as a well in a polystyrene microtiter plate. Then, a sample from a subject suspected of having a SARS-CoV-2 infection that has induced SARS-CoV-2-specific antibodies is added to the wells. After binding and/or washing to remove non-bound materials, biotinylated anti-human immunoglobulin antibodies having binding specificity for the C-region of human are introduced into the wells and allowed to bind to any human IgA or IgM antibodies bound to the SARS-CoV-2-specific targeted antigens. Detection of the presence of the IgA, IgM, and/or IgG is by reacting a streptavidin conjugated horse radish peroxidase (or other suitable enzyme) bound to the biotin moieties with a suitable substrate such as TMB. The amount of the reaction product from this substrate may be quantitatively detected by measuring its optical absorbance (for example, with TMB as the substrate at 415 nm absorbance).

As will be understood by those of ordinary skill in the art, notwithstanding individual features (e.g. the confirmatory steps described herein), in general, ELISAs have certain features in common, such as coating, incubating and binding, washing to remove non-specifically bound species, and detecting the bound immune complexes.

In coating a plate with a SARS-CoV-2-specific antigen, the wells of the plate will generally be incubated with a solution of the SARS-CoV-2-specific antigen, either overnight or for a specified period of hours. A coating buffer may be a sodium phosphate/BSA coating buffer or another suitable art-known coating buffer. The wells of the plate will then be washed to remove incompletely adsorbed material. Any remaining available surfaces of the wells may then “coated” with a nonspecific protein that is antigenically neutral with regard to the test sample. This protein may be bovine serum albumin (BSA), casein or solutions of milk powder, etc. The coating allows for blocking of non-specific adsorption sites on the immobilizing surface and thus reduces the background caused by non-specific binding of antibodies onto the surface.

In the ELISA method of the disclosure, a secondary or tertiary detection means may be used. When using a secondary or tertiary detection methods, after binding of a SARS-CoV-2-specific antigen to the well, coating with a non-reactive material to reduce background, (e.g. with blocking buffer such as Tris-sucrose blocking buffer or other art-recognized blocking buffer), and washed to remove unbound material, the immobilizing surface is contacted with the biological sample to be tested under conditions effective to allow a complex (anti-SARS-CoV-2-specific antibody/SARS-CoV-2-specific antigen) formation. Detection of this immune complex then requires a labeled (biotin) secondary binding antibody, and a third binding ligand, i.e. streptavidin-horse radish peroxidase.

The term “under conditions effective to allow immune complex formation” as used herein refers to the conditions such as, but not limited to, diluting the antigens and/or antibodies with solutions such as BSA, bovine gamma globulin (BGG), phosphate buffered saline (PBS)/Tween, PBS with casein and Tween 20, or PBS/BSA buffer with Tween 20. Various other art-known assay diluents can be used in the methods of the invention. These added agents also tend to assist in the reduction of non-specific background.

The “suitable” conditions as used herein, means that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. Various art-known assay temperature and timing parameters can be used in the methods of the invention.

To provide a detecting means, the second or third antibody will have an associated detectable label. In certain embodiments, the detectable label is an enzyme that will generate color development upon incubating with an appropriate chromogenic substrate. Thus, for example, one may contact or incubate the first and second immune complex with a urease-, glucose oxidase-, alkaline phosphatase-, hydrogen peroxidase-conjugated antibody, or other conjugated antibody for a period of time and under conditions that favor the development of further immune complex formation (e.g., incubation for 2 hours at room temperature in a PBS-containing solution such as PBS-Tween).

It also will be understood by those of skill in the art that one or more positive and negative quality controls may be utilized in the methods of the invention. A positive quality control sample may be a normal serum sample that contains a predetermined amount of a human IgA or IgM antibody. Quality control samples may be reacted in parallel with and under the same conditions as the biological and control samples of the assay and provide a measure of the function of the assay.

A negative quality control sample may be a serum sample known not to include an antibody that is known to bind to a SARS-CoV-2-specific antigen. One of ordinary skill will understand how to utilize positive and negative control reactions and samples in an ELISA to ascertain and validate the functionality of the solutions and/or substrates and/or protocol used in the assay. A positive control may include a known amount of a human IgA, IgM, or IgG antibody. A negative control may be a sample that is known to not include a human IgA, IgM, or IgG antibody. Such a negative control, when treated under the same conditions as the test sample (e.g., the biological sample), will demonstrate that the binding detected in a biological sample arises from the biological sample and is not due to contamination of assay components or other factor not associated with the biological sample.

It also is contemplated that the ELISA reagents described herein can be packaged in a kit that may be produced commercially to detect the presence of and/or measure a human IgA, IgM, or IgG antibody, which specifically binds a SARS-CoV-2-specific antigen, in a biological sample as described herein.

It should be appreciated that biological samples may be diluted before being assayed (e.g., 2 fold, 5 fold, 10 fold, 50 fold, 100 fold, and including higher or lower fold values or any fold value in between). In one embodiment, a reference sample containing a clinically significant threshold amount of reference antibody may be diluted by the same amount as the biological sample being tested so that the signal obtained for the biological sample can be compared directly to the signal obtained for the reference sample.

The methods of the disclosure can be performed in a qualitative format, which determines the presence or absence of at least one of a human IgA, IgM, and IgG antibody in the sample and which specifically binds a SARS-CoV-2-specific antigen, but preferably in a quantitative format, which, in addition, provides a measurement of the quantity present in the sample of at least one of a human IgA, IgM, and IgG that specifically binds a SARS-CoV-2-specific.

The assays of the disclosure, therefore, comprise the steps of adding a serum sample to be tested to a first multi-well plate or plates well known in the art and which has wells coated with SARS-CoV-2 N protein or antigenic fragments thereof, a SARS-CoV-2 S protein or antigenic fragments thereof or, most advantageously, a combination of SARS-CoV-2 N protein, SARS-CoV-2 S protein, either as native polypeptides and/or antigenic fragments thereof.

More advantageously, the wells of the first multi-well plate can be coated with SARS-CoV-2 N protein or antigenic fragments thereof, and a fragment of the SARS-CoV-2 S protein comprising the Receptor Binding Domain (RBD) thereof. Further provided is a second multi-well plate wherein the wells are coated with serum albumin. In some embodiments, the SARS protein coated and albumin-coated wells are of the same multi-well plate.

A diluted serum sample from a human suspected of being infected with the SARS-CoV-2 virus for sufficient time to have generated serum IgA, IgM, and/or IgG specifically binding a SARS-CoV-2-specific antigen is added to at least two wells of the plate.

The “normal” or negative controls used with these assay systems are clearly distinguished from “positive” samples. Results are reported either as a qualitative result (positive or negative), using specific mean channel cut-off or as semi-quantitative values by dividing the mean channel fluorescence of the positive sample by the mean channel of the negative control. This creates the potential for monitoring serum titers of the specific analyte. Quantitative results may also be incorporated by utilizing known multiple positive control standards, which may form concentration curves when plotted on a graph of result versus concentration value.

For the purposes of the methods of the disclosure, it is most advantageous for the SARS-CoV-2-derived antigen to be derived from the Spike protein of the virus. The full-length expressed protein has the amino acid sequence SEQ ID NO: 1 (Accession No: QHD43416) with amino acids V16-Q690 being the sequence minus a leader sequence. A most advantageous polypeptide derived from the spike protein is a fragment encompassing the Receptor Binding Domain (RBD) such as, but not limited to the amino acids R319-F541 (SEQ ID NO: 2) or antigenic fragments thereof that can have affinity with, and bound by, an anti-SARS-CoV-2-specific antibody. Determination of antigenic fragments of SEQ ID NO: 2 that can be useful in the methods of the disclosure can be obtained and confirmed to bind anti-SARS-CoV-2 antibodies by methods well-known to those of skill in the arts.

The methods of the disclosure are further most advantageously adapted by the use of the spike protein region 2 (amino acids M697-P1213 (SEQ ID NO: 3), or antigenic fragments thereof, and the amino acids M1-A419 of the SARS-CoV-2-derived nucleocapsid protein (SEQ ID NO: 4 (Accession No: QHD43423)), or antigenic fragments thereof.

The present disclosure further provides embodiments of kits for practicing the screening methods of the disclosure. By “kit” is intended any manufacture (e.g., a package or a container) comprising at least one reagent for specifically detecting the presence of a SARS-CoV-2-specific antibody in a sample from a human or animal subject. The kit may be promoted, distributed, or sold as a unit for performing the methods of the present invention. Additionally, the kits may contain a package insert describing the kit and methods for its use.

In some embodiments, kits are for use in screening for identifying patients with at least one anti-SARS-CoV-2 antibody. Chemicals for the detection of anti-SARS-CoV-2 antibody binding to the bead-bound SARS-CoV-2 antigen by multiplex bead-based immunoassay format may be further included in a kit of the disclosure.

One of skill in the art will further appreciate that any or all steps in the screening methods of the invention could be implemented by personnel or, alternatively, performed in an automated fashion. That is, the methods can be performed in an automated, semi-automated, or manual fashion. Furthermore, the methods disclosed herein can also be combined with other methods known or later developed to permit a more accurate identification of patients having a SARS-CoV-2 infection or having been exposed to the SARS-CoV-2 virus.

The disclosure provides embodiments of an ELISA method readily adapted for use as a high-throughput high-sensitivity method, and kits to enable the method to detect human IgA, IgM, and IgG antibodies directed against SARS-CoV-2 spike and nucleocapsid-specific antigens, the simultaneous use of the spike- and N (nucleocapsid)-derived targets resulting in surprisingly few false positive results compared to existing ELISA-based methods.

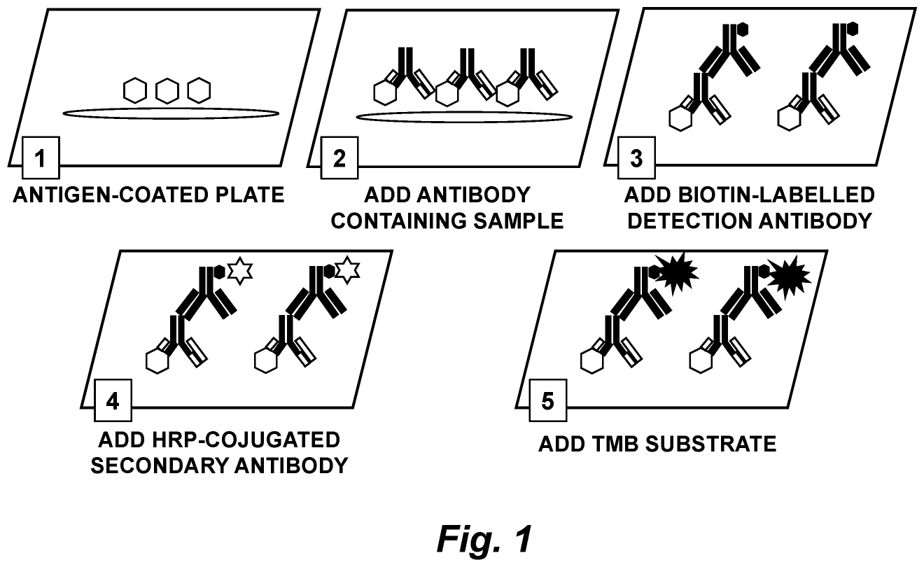

The assay method of the disclosure is schematically shown in FIG. 1,

One aspect of the disclosure encompasses embodiments of a method of detecting an immune response to SARS-CoV-2, the method comprising: (a) incubating, under conditions effective to allow immune complex formation, a serum sample from a subject suspected of having been exposed to a SARS-CoV-2 virus with a mixture of a SARS-CoV-2 spike protein, or fragments thereof, and a SARS-CoV-2 nucleocapsid protein, or fragments thereof, wherein the mixture of SARS-CoV-2 N and S-derived proteins or fragments thereof is bound to a plurality of wells of a multi-well plate; (b) incubating, under conditions effective to allow immune complex formation, the serum sample from the subject with a serum albumin, wherein the serum albumin is bound to a control well of a multi-well plate; (c) washing unbound serum samples from the wells; (d) adding a biotinylated anti-human IgA antibody, a biotinylated anti-human IgM, or a biotinylated anti-human IgG antibody, or biotinylated antigen-binding fragments thereof, to the well having the bound SARS-CoV-2 proteins or fragments thereof, and to the control well; (e) incubating the wells for a period effective to allow the anti-human Ig antibody, or antigen-binding fragments thereof, to bind to serum anti-SARS-CoV-2 antibodies bound to the SARS-CoV-2 proteins or fragments thereof, and washing the wells to remove unbound biotinylated anti-human Ig antibody; (f) adding a horse radish peroxidase (HRP)-streptavidin conjugate and an HRP substrate to each of the wells from step (e), thereby generating a light detectable product; (g) determining the light absorbance of the product from step (f) for each of the wells; (h) subtracting the absorbance measured for the control well from the absorbance of the well having the bound SARS-CoV-2 proteins or fragments thereof; and (i) calculating the amount of Ig in the serum sample from the measured absorbance of the well having the bound SARS-CoV-2 proteins or fragments thereof minus the measured absorbance of the control well.

In some embodiments of this aspect of the disclosure, the method can further comprise repeating the step (d) for each of the biotinylated anti-human IgA antibody, the biotinylated anti-human IgM antibody, and the biotinylated anti-human IgG antibody, or the antigen-binding fragments thereof.

In some embodiments of this aspect of the disclosure, the method can further comprise determining the relative levels of at least two of human IgA, IgM, and IgG bound to the SARS-CoV-2 proteins or fragments thereof, thereby determining the immune response of the subject to a SARS-CoV-2 infection.

In some embodiments of this aspect of the disclosure, the HRP substrate can be 3,3,5,5′-tetramethylbenzidine.

In some embodiments of this aspect of the disclosure, each of the wells can be of a single multi-well plate.

In some embodiments of this aspect of the disclosure, the wells receiving the anti-human Ig antibody can be of different multi-well plates.

In some embodiments of this aspect of the disclosure, the method can be a high-throughput assay.

In some embodiments of this aspect of the disclosure, the bound SARS-CoV-2 can comprise the SARS-CoV-2 spike protein from amino acid positions Arg319 to Phe541 (SEQ ID NO: 2) of the receptor-binding domain (RBD).

Another aspect of the disclosure encompasses embodiments of a kit comprising: (i) a vessel or vessels containing at least one of a biotinylated anti-human IgA antibody, a biotinylated anti-human IgM antibody, and a biotinylated anti-human IgG antibody, or antigen-binding fragments thereof; (ii) at least one multi-well plate comprising a plurality of wells, wherein the wells are coated with a mixture of a SARS-CoV-2 spike protein, or a fragment thereof, and a SARS-CoV-2 nucleocapsid protein, or fragments thereof; (iii) a vessel containing a horse radish peroxidase-streptavidin conjugate; and (iv) instructions for the use of the reagents of the kit in the method of claim 1 for the detection of at least one of aSARS-CoV-2-specific IgA, IgM or IgG antibody.

In some embodiments of this aspect of the disclosure, the protein of SARS-CoV-2 is a SARS-CoV-2 spike protein.

In some embodiments of this aspect of the disclosure, the protein of SARS-CoV-2 comprises the SARS-CoV-2 spike protein from amino acid positions Arg319 to Phe541 (SEQ ID NO: 2) of the receptor-binding domain (RBD).

As mentioned above, compounds of the present disclosure and pharmaceutical compositions can be used in combination of one or more other therapeutic agents for treating viral infection and other diseases. For example, compounds of the present disclosure and pharmaceutical compositions provided herein can be employed in combination with other anti-viral agents to treat viral infection.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

### Kit Components for the Detection of Anti-SARS-CoV Antigen IgA

TABLE 1

| | | Storage/Stability |
|---|---|---|
| Component | Size/Description | After Preparation |
| SARS-CoV-2 N and S1 | 96 wells (12 strips × 8 wells) coated with | 1 month at 4° C.* |
| RBD protein coated 96 | SARS-CoV-2 N and S1 RBD protein | |
| well-Microplate (Item A) | | |
| Albumin protein coated 96 | 96 wells (12 strips × 8 wells) coated with | 1 month at 4° C.* |
| well-Microplate (Item D) | Albumin protein | |
| Wash Buffer Concentrate | 40 ml of 20x concentrated solution. | 1 month at 4° C. |
| (20X) (Item B) | | |
| Positive Control (Item C) | 2 vials of Positive Control sample from an | 1 week at −80° C. |
| | inactivated serum sample which contains | |
| | SARS-Cov-2 N and S1 RBD protein | |
| | human IgA antibody. | |
| Biotinylated Anti-Human | 2 vials of solution. | 5 days at 4° C. |
| IgA (Item F) | | |
| HRP-Streptavidin | 1 vial of solution. | Do not store and |
| concentrate (Item G) | | reuse |
| TMB One-Step Substrate | 24 ml of 3,3,5,5′-tetramethylbenzidine | 1 month at 4° C. |
| Reagent (Item H) | (TMB buffer solution). | |
| Stop Solution (Item I) | 16 ml of 0.2M sulfuric acid. | N/A |
| Assay Diluent B (Item E) | 15 ml of 5x concentrated buffer. | 1 month at 4° C. |
| 5x Sample Diluent (Item J) | 25 ml of 5x diluent buffer, 0.5% proclin | 1 month at 4° C. |
| | 300 as preservative. | |

### Example 2

### Reagent Preparation

1. Reagents and samples are brought to room temperature (18-25° C.) before use.
2. 5× Sample Diluent (Item J) is diluted 5-fold with deionized or distilled water before use to make 1× Sample Diluent.
3. 5× Assay Diluent B (Item E) is diluted 5-fold with deionized or distilled water before use to make 1× Assay Diluent B.
4. Dilute a human serum sample 1:1500 with 1× Sample Diluent (Item J). For example, add 1 μl serum to 1499 μl 1× Sample Diluent and mix.

Avoid samples showing severe hemolysis, precipitate, contamination by bacteria, or protein suspension.

EDTA, heparin sulfate, sodium citrate, or other anti-coagulants does affect the results.

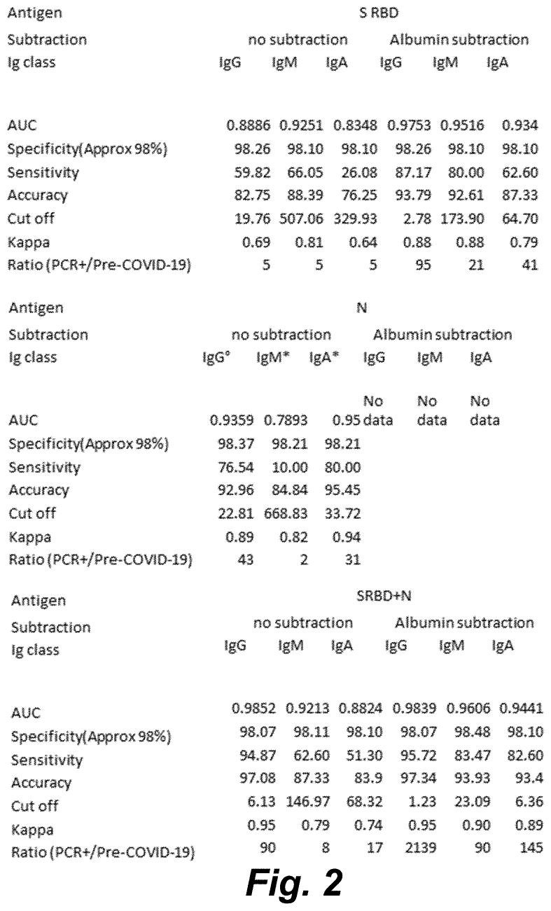

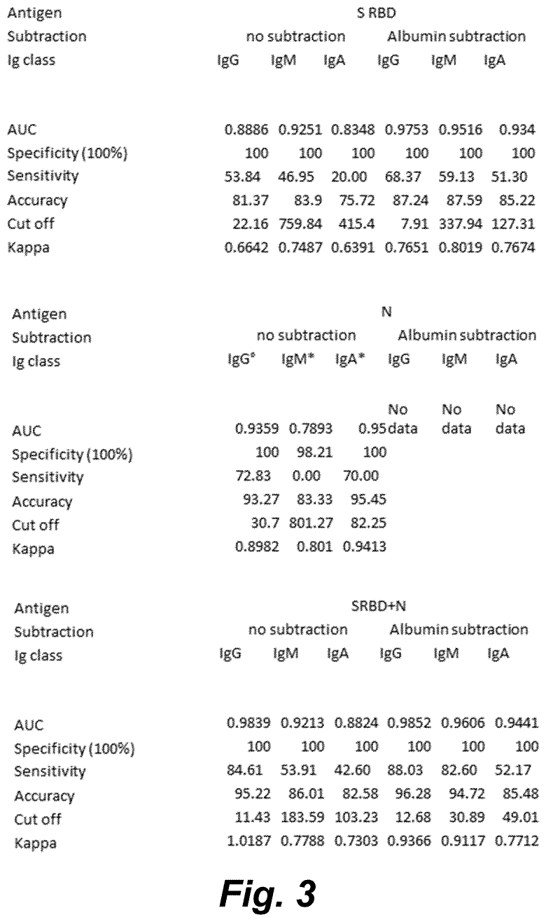

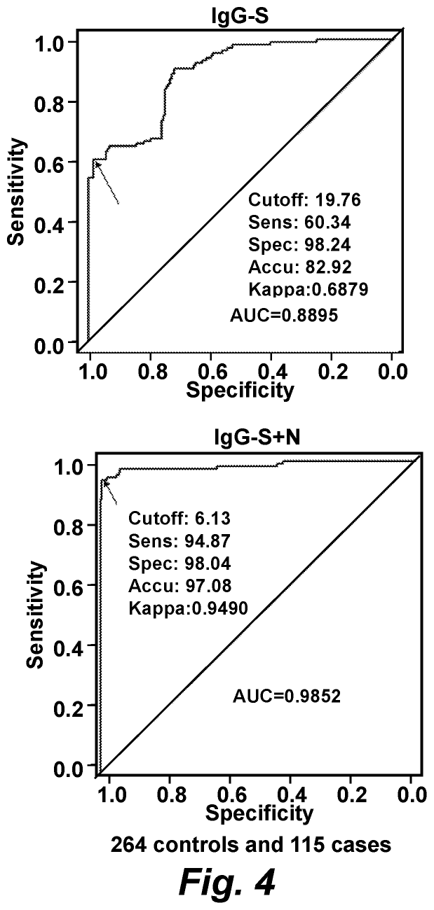

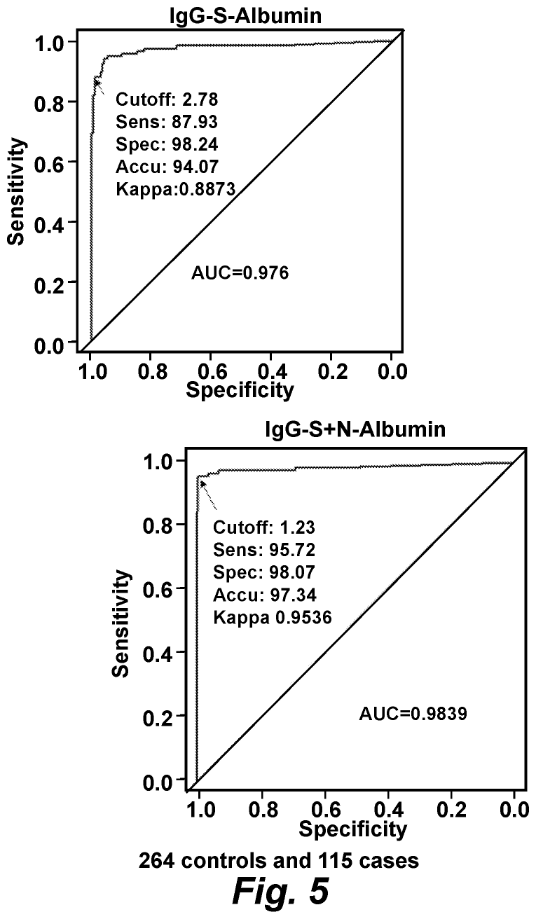

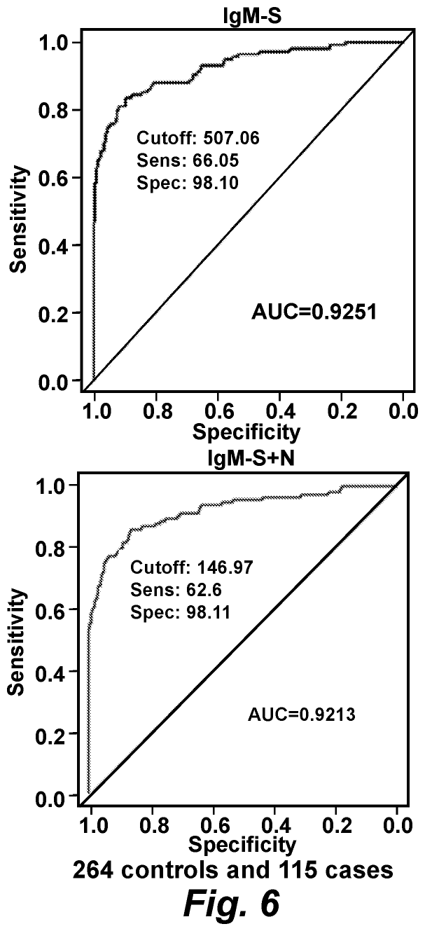

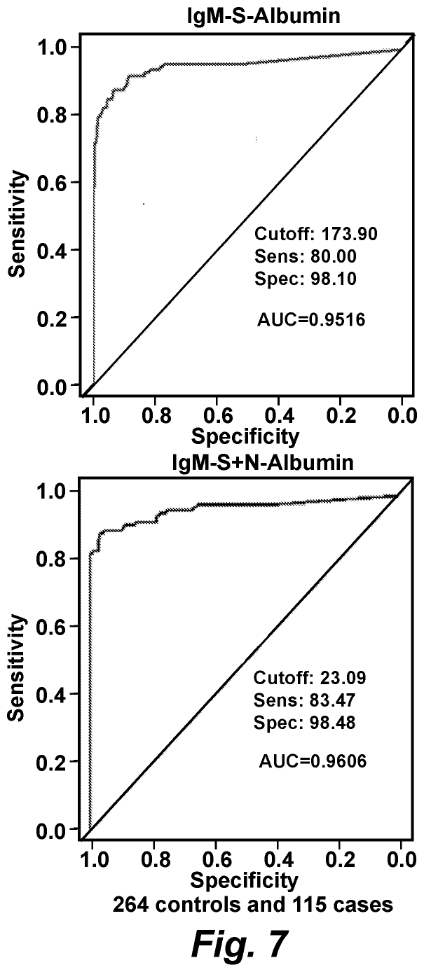

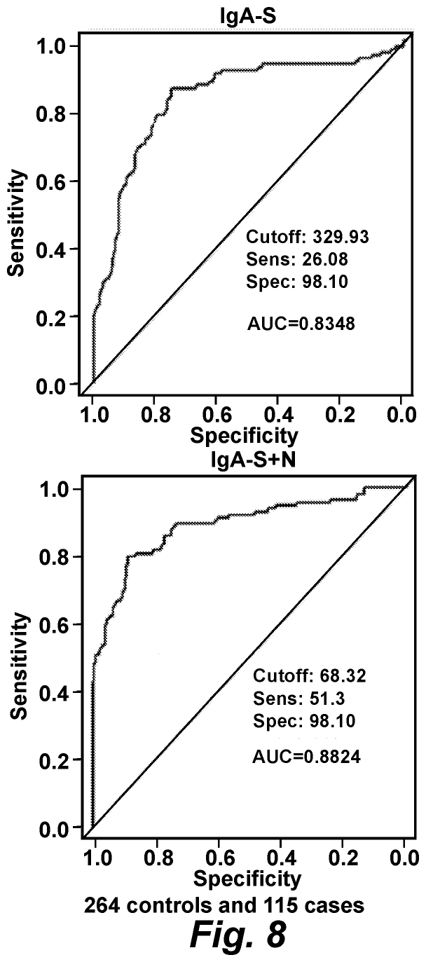

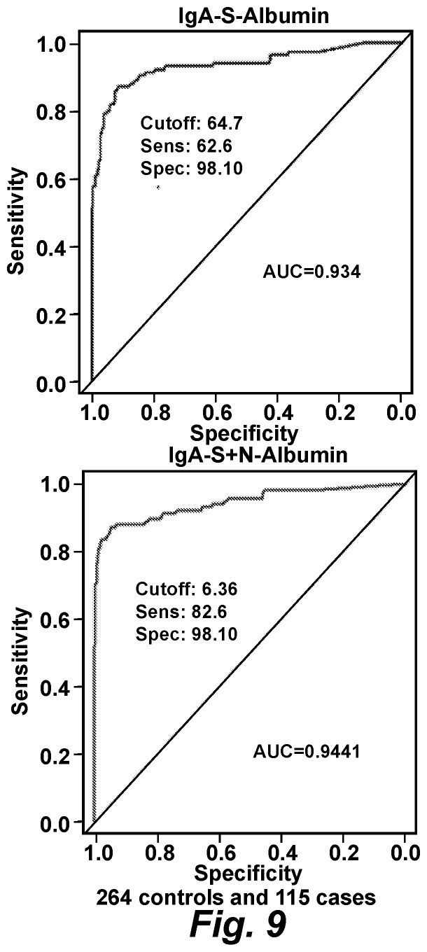

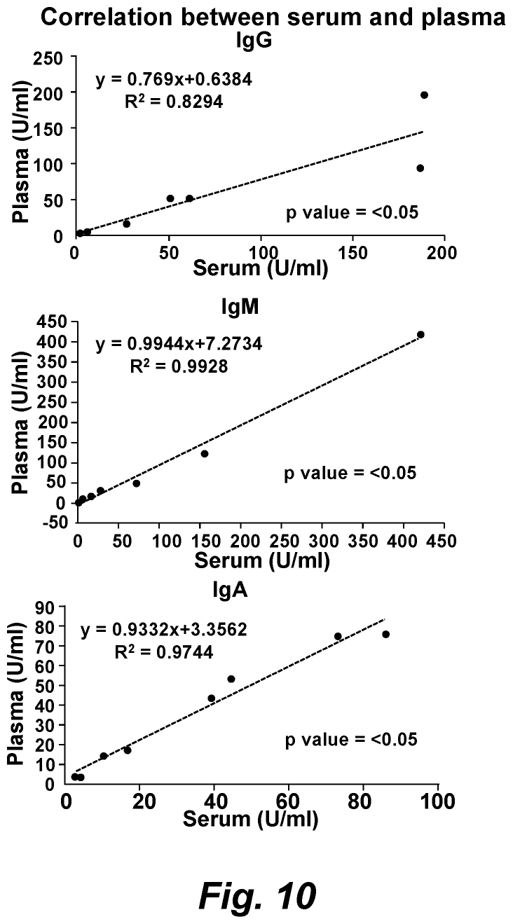

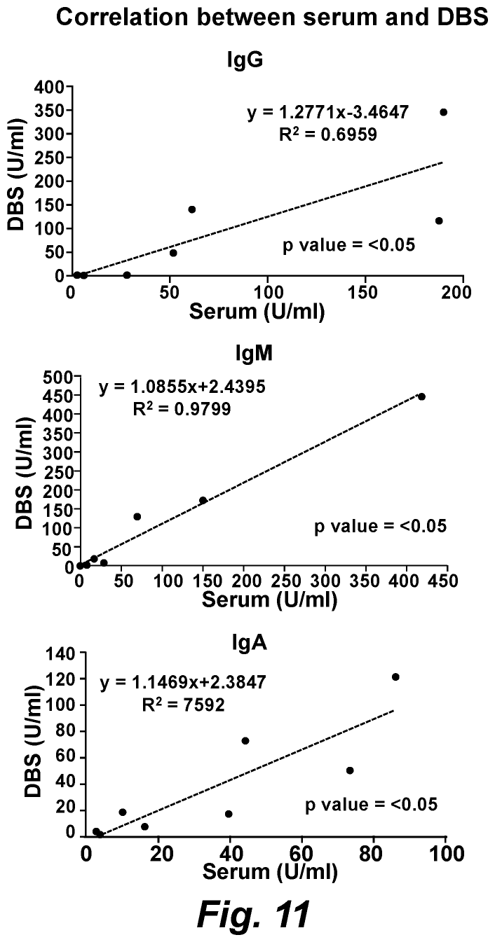

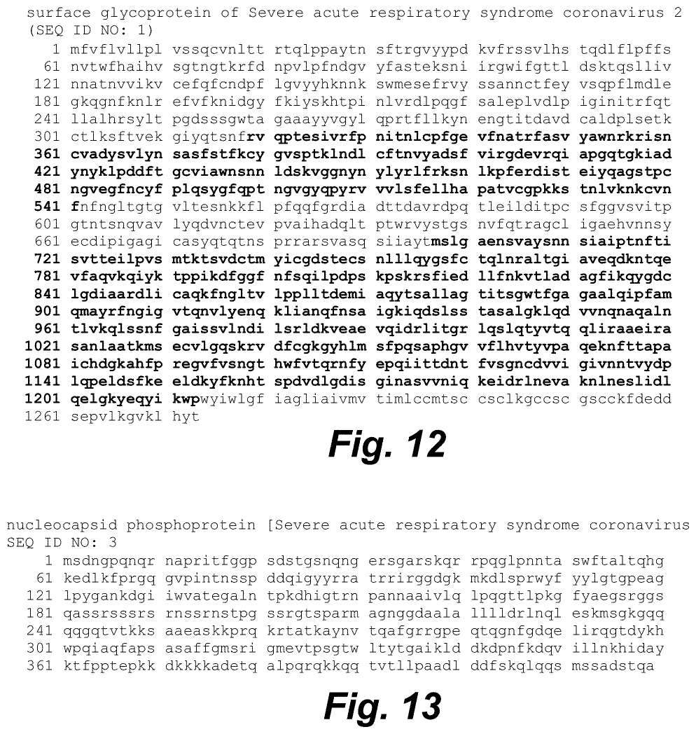

5. Briefly spin the vials of Positive Control, Item C
6. Add 400 μl 1× Sample Diluent (Item J) into each Item C vial to prepare a 1000 Unit/ml Positive Control solution and mix thoroughly.
7. Pipette 320 μl 1× Sample Diluent into 2 sets each of 7 tubes. Use the 1000 Unit/ml Positive Control solution to produce a dilution series as shown in FIG. 2. Mix each tube thoroughly before the next transfer. 1× Sample Diluent serves as the zero (0 Unit/ml).
8. If the Wash Concentrate (20×) (Item B) contains visible crystals, warm to room temperature and mix gently until dissolved. Dilute 40 ml of Wash Buffer Concentrate into deionized or distilled water to give 800 ml of 1× Wash Buffer.
9. Briefly spin the biotinylated anti-human IgA antibody vial (Item F) before use. Add 200 μl of 1× Assay Diluent B (Item E) into each vial to prepare an antibody concentrate. Pipette mix gently (the concentrate can be stored at 4° C. for 5 days). The detection antibody concentrate should then be diluted 100-fold with 1× Assay Diluent B and used in step 5 of the Assay Procedure (Example 3 of the disclosure).
10. Briefly spin the HRP-Streptavidin concentrate (Item G) and pipette mix gently before use. HRP-streptavidin concentrate is diluted 800-fold with 1× Assay Diluent B (Item E) and used in step 7 of the Assay Procedure (Example 3 of the disclosure).

For example: Briefly spin the vial (Item G) and pipette mix gently. Add 25 μl of HRP-Streptavidin concentrate per tube with 20 ml 1× Assay Diluent B to prepare a 800-fold diluted HRP-streptavidin solution (do not store the diluted solution for next day use). Mix well.

### Example 3

### Assay Procedure

1. Bring all reagents and samples to room temperature (18-25° C.) before use. The positive control and all samples should be run at least in duplicate.
2. Label removable 8-well strips as appropriate.
3. Add 100 μl of each prepared positive control (Item C, prepared in Reagent Preparation step 5), and sample (prepared in Reagent Preparation step 4) into appropriate wells of the SARS-CoV-2 N and S1 RBD protein coated 96 well-Microplate (Item A) and the albumin protein coated 96 well-Microplate (Item D). Cover wells and incubate for 1 hr at room temperature with gentle shaking.
4. Discard the solution and wash 4 times with 1× Wash Buffer. Wash by filling each well with 300 μl of 1× Wash Buffer using a multi-channel Pipette or autowasher. Complete removal of all liquid at each step is essential for good performance. After the last wash, remove any remaining Wash Buffer by aspirating or decanting. Invert the plate and blot it against clean paper towels.
5. Add 100 μl of prepared biotinylated anti-Human IgA antibody (Item F, Reagent Preparation step 7) to each well. Incubate for 30 mins at room temperature with gentle shaking.
6. Discard the solution. Repeat the wash as in step 4.
7. Add 100 μl of prepared HRP-Streptavidin solution (see Reagent Preparation step 8) to each well. Incubate for 30 mins at room temperature with gentle shaking.
8. Discard the solution. Repeat the wash as in step 4.
9. Add 100 μl of TMB One-Step Substrate Reagent (Item H) to each well. Incubate for 15 mins at room temperature in the dark with gentle shaking.
10. Add 50 μl of Stop Solution (Item I) to each well. Measure the absorbance at 450 nm immediately.

### Assay Procedure Summary

1. Prepare all reagents, samples and standards as in Example 2.
2. Add 100 μl positive control, or sample to each well. Incubate 1 hr at room temperature.
3. Add 100 μl prepared biotinylated anti-Human IgA antibody into each well. Incubate 30 mins at room temperature.
4. Add 100 μl prepared HRP-streptavidin solution to each well. Incubate 30 mins at room temperature.
5. Add 100 μl TMB One-Step Substrate Reagent to each well. Incubate 15 mins at room temperature.
6. Add 50 μl Stop Solution to each well. Measure the absorbance at 450 nm immediately.

### Example 4

### Interpretation of Results

1. Subtract the signals of all wells of the albumin protein-coated plate from the signals of all wells of the N and S1 RBD coated plate, including positive control and samples, to remove the background.
2. Calibration curve: Calculate the mean absorbance for each set of duplicate Positive Control (Item C), and samples from the background subtracted N and S1 RBD plate and then subtract the average zero Positive Control optical density. Plot the calibration curve on a log-log scale with Positive Control concentration (Unit/ml) on the x-axis and absorbance on the y-axis using SIgAa plot or Excel software. A calibration curve must be run with each assay.
3. A positive result for an unknown sample is considered as a Unit/ml calculated value using a calibration curve of greater than 6.36 Unit/ml.
4. A negative result for an unknown sample is considered as a Unit/ml calculated value using the calibration curve of less than 6.36 Unit/ml.

### Example 5

### Assay Performance

1. The sensitivity of this assay against a reference standard is 82.60% (95/115, 95% Cl: 74.42-89.04%).
2. The specificity of this assay against a reference standard is 98.10% (259/264, 95% Cl: 95.63-99.38%).
3. The accuracy of this assay against a reference standard is 93.40. % (354/379, 95% Cl: 90.41%-95.68%), with Kappa value of 0.8887 and the AUC=0.9441.

### Example 6

### Kit Components for the Detection of Anti-SARS-Co V Antigen IgM

TABLE 2

| | | Storage/Stability |
|---|---|---|
| Component | Size/Description | After Preparation |
| SARS-CoV-2 N and S1 | 96 wells (12 strips × 8 wells) coated with | 1 month at 4° C.* |
| RBD protein coated 96 well- | SARS-CoV-2 N and S1 RBD protein | |
| Microplate (Item A) | | |
| Albumin protein coated 96 | 96 wells (12 strips × 8 wells) coated with | 1 month at 4° C.* |
| well-Microplate (Item D) | Albumin protein | |
| Wash Buffer Concentrate | 40 ml of 20x concentrated solution. | 1 month at 4° C. |
| (20x) (Item B) | | |
| Positive Control (Item C) | 2 vials of Positive Control sample from an | 1 week at −80° C. |
| | inactivated serum sample which contains | |
| | SARS-Cov-2 N and S1 RBD protein | |
| | human IgM antibody. | |
| Biotinylated Anti-Human | 2 vials of solution. | 5 days at 4° C. |
| IgM (Item F) | | |
| HRP-Streptavidin | 1 vial of solution. | Do not store and |
| concentrate (Item G) | | reuse |
| TMB One-Step Substrate | 24 ml of 3,3,5,5′-tetramethylbenzidine | 1 month at 4° C. |
| Reagent (Item H) | (TMB buffer solution). | |
| Stop Solution (Item I) | 16 ml of 0.2M sulfuric acid. | N/A |
| Assay Diluent B (Item E) | 15 ml of 5x concentrated buffer. | 1 month at 4° C. |
| 5x Sample Diluent (Item J) | 25 ml of 5x diluent buffer, 0.5% proclin 300 | 1 month at 4° C. |
| | as preservative. | |

*Return unused wells to the pouch containing desiccant pack, reseal along entire edge.

### Example 7

### Reagent Preparation

1. Reagents and samples are brought to room temperature (18-25° C.) before use.
2. 5× Sample Diluent (Item J) is diluted 5-fold with deionized or distilled water before use to make 1× Sample Diluent.
3. 5× Assay Diluent B (Item E) is diluted 5-fold with deionized or distilled water before use to make 1× Assay Diluent B.
4. Dilute a human serum sample 1:1500 with 1× Sample Diluent (Item J). For example, add 1 μl serum to 1499 μl 1× Sample Diluent and mix.

Avoid samples showing severe hemolysis, precipitate, contamination by bacteria, or protein suspension.

EDTA, heparin sulfate, sodium citrate, or other anti-coagulants does affect the results.

5. Briefly spin the vials of Positive Control, Item C.
6. Add 400 μl 1× Sample Diluent (Item J) into each Item C vial to prepare a 1000 Unit/ml Positive Control solution and mix thoroughly.
7. Pipette 320 μl 1× Sample Diluent into 2 sets each of 7 tubes. Use the 1000 Unit/ml Positive Control solution to produce a dilution series as shown in FIG. 2. Mix each tube thoroughly before the next transfer. 1× Sample Diluent serves as the zero (0 Unit/ml).
8. If the Wash Concentrate (20×) (Item B) contains visible crystals, warm to room temperature and mix gently until dissolved. Dilute 40 ml of Wash Buffer Concentrate into deionized or distilled water to give 800 ml of 1× Wash Buffer.
9. Briefly spin the biotinylated anti-human IgM antibody vial (Item F) before use. Add 200 μl of 1× Assay Diluent B (Item E) into each vial to prepare an antibody concentrate. Pipette mix gently (the concentrate can be stored at 4° C. for 5 days). The detection antibody concentrate should then be diluted 100-fold with 1× Assay Diluent B and used in step 5 of the Assay Procedure (Example 3 of the disclosure).
10. Briefly spin the HRP-Streptavidin concentrate (Item G) and pipette mix gently before use. HRP-streptavidin concentrate is diluted 800-fold with 1× Assay Diluent B (Item E) and used in step 7 of the Assay Procedure (Example 3 of the disclosure).

For example: Briefly spin the vial (Item G) and pipette mix gently. Add 25 μl of HRP-Streptavidin concentrate per tube with 20 ml 1× Assay Diluent B to prepare a 800-fold diluted HRP-streptavidin solution (do not store the diluted solution for next day use). Mix well.

### Example 8

### Assay Procedure

Bring all reagents and samples to room temperature (18-25° C.) before use. The positive control and all samples should be run at least in duplicate.

Label removable 8-well strips as appropriate.

Add 100 μl of each prepared positive control (Item C, prepared in Reagent Preparation step 5), and sample (prepared in Reagent Preparation step 4) into appropriate wells of the SARS-CoV-2 N and 51 RBD protein coated 96 well-Microplate (Item A) and the albumin protein coated 96 well-Microplate (Item D). Cover wells and incubate for 1 hr at room temperature with gentle shaking.

Discard the solution and wash 4 times with 1× Wash Buffer. Wash by filling each well with 300 μl of 1× Wash Buffer using a multi-channel Pipette or autowasher. Complete removal of all liquid at each step is essential for good performance. After the last wash, remove any remaining Wash Buffer by aspirating or decanting. Invert the plate and blot it against clean paper towels.

Add 100 μl of prepared biotinylated anti-Human IgM antibody (Item F, Reagent Preparation step 7) to each well. Incubate for 30 mins at room temperature with gentle shaking.

Discard the solution. Repeat the wash as in step 4.

Add 100 μl of prepared HRP-Streptavidin solution (see Reagent Preparation step 8) to each well. Incubate for 30 mins at room temperature with gentle shaking.

Discard the solution. Repeat the wash as in step 4.

Add 100 μl of TMB One-Step Substrate Reagent (Item H) to each well. Incubate for 15 mins at room temperature in the dark with gentle shaking.

Add 50 μl of Stop Solution (Item I) to each well. Measure the absorbance at 450 nm immediately.

### Assay Procedure Summary

Prepare all reagents, samples and standards as in Example 2.

Add 100 μl positive control, or sample to each well. Incubate 1 hr at room temperature.

Add 100 μl prepared biotinylated anti-Human IgA antibody into each well. Incubate 30 mins at room temperature.

Add 100 μl prepared HRP-streptavidin solution to each well. Incubate 30 mins at room temperature.

Add 100 μl TMB One-Step Substrate Reagent to each well. Incubate 15 mins at room temperature.

Add 50 μl Stop Solution to each well. Measure the absorbance at 450 nm immediately.

### Example 9

### Interpretation of Results

1. Subtract the signals of all wells of the albumin protein-coated plate from the signals of all wells of the N and 51 RBD coated plate, including positive control and samples, to remove the background.
2. Calibration curve: Calculate the mean absorbance for each set of duplicate Positive Control (Item C), and samples from the background subtracted N and 51 RBD plate and then subtract the average zero Positive Control optical density. Plot the calibration curve on a log-log scale with Positive Control concentration (Unit/ml) on the x-axis and absorbance on the y-axis using SIgAa plot or Excel software. A calibration curve must be run with each assay.
3. A positive result for an unknown sample is considered as a Unit/ml calculated value using a calibration curve of greater than 23.09 Unit/ml.
4. A negative result for an unknown sample is considered as a Unit/ml calculated value using the calibration curve of less than 23.09 Unit/ml.

### Example 10

### Assay Performance

1. The sensitivity of this assay against a reference standard is 83.47% (96/115, 95% Cl: 75.4%-89.74%).
2. The specificity of this assay against a reference standard is 98.48% (260/264, 95% Cl: 96.16-99.58%).
3. The accuracy of this assay against a reference standard is 93.93% (356/379, 95% Cl: 91.03%-96.11%), with Kappa value of 0.8976 and the AUC=0.9606.

### Kit Components for the Detection of Anti-SARS-CoV Antigen IgG

TABLE 3

| | | Storage/Stability |
|---|---|---|
| Component | Size/Description | After Preparation |
| SARS-CoV-2 N and S1 | 96 wells (12 strips × 8 wells) coated with | 1 month at 4° C.* |
| RBD protein coated 96 well- | SARS-CoV-2 N and S1 RBD protein | |
| Microplate (Item A) | | |
| Albumin protein coated 96 | 96 wells (12 strips × 8 wells) coated with | 1 month at 4° C.* |
| well-Microplate (Item D) | Albumin protein | |
| Wash Buffer Concentrate | 40 ml of 20x concentrated solution. | 1 month at 4° C. |
| (20x) (Item B) | | |
| Positive Control (Item C) | 2 vials of Positive Control sample from an | 1 week at −80° C. |
| | inactivated serum sample which contains | |
| | SARS-Cov-2 N and S1 RBD protein | |
| | human IgM antibody. | |
| Biotinylated Anti-Human | 2 vials of solution. | 5 days at 4° C. |
| IgG (Item F) | | |
| HRP-Streptavidin | 1 vial of solution. | Do not store and |
| concentrate (Item G) | | reuse |
| TMB One-Step Substrate | 24 ml of 3,3,5,5′-tetramethylbenzidine | 1 month at 4° C. |
| Reagent (Item H) | (TMB buffer solution). | |
| Stop Solution (Item I) | 16 ml of 0.2M sulfuric acid. | N/A |
| Assay Diluent B (Item E) | 15 ml of 5x concentrated buffer. | 1 month at 4° C. |
| 5x Sample Diluent (Item J) | 25 ml of 5x diluent buffer, 0.5% proclin 300 | 1 month at 4° C. |
| | as preservative. | |

*Return unused wells to the pouch containing desiccant pack, reseal along entire edge.
*Return unused wells to the pouch containing desiccant pack, reseal along entire edge.

### Example 12

### Reagent Preparation

1. Reagents and samples are brought to room temperature (18-25° C.) before use.
2. 5× Sample Diluent (Item J) is diluted 5-fold with deionized or distilled water before use to make 1× Sample Diluent.
3. 5× Assay Diluent B (Item E) is diluted 5-fold with deionized or distilled water before use to make 1× Assay Diluent B.
4. Dilute a human serum sample 1:1500 with 1× Sample Diluent (Item J). For example, add 1 μl serum to 1499 μl 1× Sample Diluent and mix.

Avoid samples showing severe hemolysis, precipitate, contamination by bacteria, or protein suspension.

EDTA, heparin sulfate, sodium citrate, or other anti-coagulants does affect the results.

5. Briefly spin the vials of Positive Control, Item C.
6. Add 400 μl 1× Sample Diluent (Item J) into each Item C vial to prepare a 1000 Unit/ml Positive Control solution and mix thoroughly.
7. Pipette 320 μl 1× Sample Diluent into 2 sets each of 7 tubes. Use the 1000 Unit/ml Positive Control solution to produce a dilution series as shown in FIG. 2. Mix each tube thoroughly before the next transfer. 1× Sample Diluent serves as the zero (0 Unit/ml).
8. If the Wash Concentrate (20×) (Item B) contains visible crystals, warm to room temperature and mix gently until dissolved. Dilute 40 ml of Wash Buffer Concentrate into deionized or distilled water to give 800 ml of 1× Wash Buffer.
9. Briefly spin the biotinylated anti-human IgG antibody vial (Item F) before use. Add 200 μl of 1× Assay Diluent B (Item E) into each vial to prepare an antibody concentrate. Pipette mix gently (the concentrate can be stored at 4° C. for 5 days). The detection antibody concentrate should then be diluted 100-fold with 1× Assay Diluent B and used in step 5 of the Assay Procedure (Example 3 of the disclosure).
10. Briefly spin the HRP-Streptavidin concentrate (Item G) and pipette mix gently before use. HRP-streptavidin concentrate is diluted 800-fold with 1× Assay Diluent B (Item E) and used in step 7 of the Assay Procedure (Example 3 of the disclosure).

For example: Briefly spin the vial (Item G) and pipette mix gently. Add 25 μl of HRP-Streptavidin concentrate per tube with 20 ml 1× Assay Diluent B to prepare a 800-fold diluted HRP-streptavidin solution (do not store the diluted solution for next day use). Mix well.

### Example 13

### Assay Procedure

Bring all reagents and samples to room temperature (18-25° C.) before use. The positive control and all samples should be run at least in duplicate.

Label removable 8-well strips as appropriate.

Add 100 μl of each prepared positive control (Item C, prepared in Reagent Preparation step 5), and sample (prepared in Reagent Preparation step 4) into appropriate wells of the SARS-CoV-2 N and S1 RBD protein coated 96 well-Microplate (Item A) and the albumin protein coated 96 well-Microplate (Item D). Cover wells and incubate for 1 hr at room temperature with gentle shaking.

Discard the solution and wash 4 times with 1× Wash Buffer. Wash by filling each well with 300 μl of 1× Wash Buffer using a multi-channel Pipette or autowasher. Complete removal of all liquid at each step is essential for good performance. After the last wash, remove any remaining Wash Buffer by aspirating or decanting. Invert the plate and blot it against clean paper towels.

Add 100 μl of prepared biotinylated anti-Human IgG antibody (Item F, Reagent Preparation step 7) to each well. Incubate for 30 mins at room temperature with gentle shaking.

Discard the solution. Repeat the wash as in step 4.

Add 100 μl of prepared HRP-Streptavidin solution (see Reagent Preparation step 8) to each well. Incubate for 30 mins at room temperature with gentle shaking.

Discard the solution. Repeat the wash as in step 4.

Add 100 μl of TMB One-Step Substrate Reagent (Item H) to each well. Incubate for 15 mins at room temperature in the dark with gentle shaking.

Add 50 μl of Stop Solution (Item I) to each well. Measure the absorbance at 450 nm immediately.

### Assay Procedure Summary

Prepare all reagents, samples and standards as in Example 2.

Add 100 μl positive control, or sample to each well. Incubate 1 hr at room temperature.

Add 100 μl prepared biotinylated anti-Human IgG antibody into each well. Incubate 30 mins at room temperature.

Add 100 μl prepared HRP-streptavidin solution to each well. Incubate 30 mins at room temperature.

Add 100 μl TMB One-Step Substrate Reagent to each well. Incubate 15 mins at room temperature.

Add 50 μl Stop Solution to each well. Measure the absorbance at 450 nm immediately.

### Example 14

### Interpretation of Results

1. Subtract the signals of all wells of the albumin protein-coated plate from the signals of all wells of the N and S1 RBD coated plate, including positive control and samples, to remove the background.
2. Calibration curve: Calculate the mean absorbance for each set of duplicate Positive Control (Item C), and samples from the background subtracted N and S1 RBD plate and then subtract the average zero Positive Control optical density. Plot the calibration curve on a log-log scale with Positive Control concentration (Unit/ml) on the x-axis and absorbance on the y-axis using SIgAa plot or Excel software. A calibration curve must be run with each assay.
A positive result for an unknown sample is considered as a Unit/ml calculated value using a calibration curve of greater than 1.23 Unit/ml.
4. A negative result for an unknown sample is considered as a Unit/ml calculated value using the calibration curve of less than 1.23 Unit/ml.

### Example 15

### Assay Performance

1. The sensitivity of this assay against a reference standard is 95.72% (112/117, 95% Cl: 90.3-98.59%).
2. The specificity of this assay against a reference standard is 98.07% (255/260, 95% Cl: 95.56-99.37%).
3. The accuracy of this assay against a reference standard is 97.34% (367/377, 95% Cl: 95.17-98.72%), with Kappa value of 0.9536 and the AUC=0.9839

### Example 16

TABLE 4
Comparison of false positive and negative for SRBD and N IgG, IgM and IgA

| | | # of | # of | | # of | # of |
|---|---|---|---|---|---|---|
| | | samples as | similarly of | | samples as | similarly of |
| | Specificity | FP | FP | Sensitivity | FN | FN |
| IgG | 98.07% | 4 | 0 | 95.72% | 5 | 4 |
| IgM | 98.48% | 4 | 0 | 83.47% | 19 | 4 |
| IgA | 98.1% | 5 | 0 | 82.6% | 20 | 4 |
| S1RBD | 98.10% | 5 | 0 | 66.05% | 39 | 1 |
| N | 98.21% | 1 | 0 | 10% | 9 | 1 |
| S1RBD | 98.10% | 5 | 0 | 26.08% | 85 | 2 |
| N | 98.21% | 1 | 0 | 80% | 2 | 2 |

What is claimed:

1. A method of detecting an immune response to SARS-CoV-2, the method comprising:
(a) incubating, under conditions effective to allow immune complex formation, a serum sample from a subject suspected of having been exposed to a SARS-CoV-2 virus with a mixture of a SARS-CoV-2 spike protein, or fragments thereof, and a SARS-CoV-2 nucleocapsid protein, or fragments thereof, wherein the mixture of SARS-CoV-2 proteins or fragments thereof are bound to a plurality of wells of a multi-well plate;
(b) incubating, under conditions effective to allow immune complex formation, the serum sample from the subject with a serum albumin, wherein the serum albumin is bound to a control well of a multi-well plate;
(c) washing unbound serum samples from the wells;
(d) adding a biotinylated anti-human IgA antibody, a biotinylated anti-human IgM, or a biotinylated anti-human IgG antibody, or biotinylated antigen-binding fragments thereof, to the well having the bound SARS-CoV-2 proteins or fragments thereof, and to the control well;
(e) incubating the wells for a period effective to allow the anti-human Ig antibody, or antigen-binding fragments thereof, to bind to serum anti-SARS-CoV-2 antibodies bound to the SARS-CoV-2 proteins or fragments thereof, and washing the wells to remove unbound biotinylated anti-human Ig antibody;
(f) adding a horse radish peroxidase (HRP)-streptavidin conjugate and an HRP substrate to each of the wells from step (e), thereby generating a light detectable product;
(g) determining the light absorbance of the product from step (f) for each of the wells;
(h) subtracting the absorbance measured for the control well from the absorbance of the well having the bound SARS-CoV-2 proteins or fragments thereof; and
(i) calculating the amount of Ig in the serum sample from the measured absorbance of the well having the bound SARS-CoV-2 proteins or fragments thereof minus the measured absorbance of the control well.

2. The method of claim 1, further comprising repeating the step (d) for each of the biotinylated anti-human IgA antibody, the biotinylated anti-human IgM antibody, and the biotinylated anti-human IgG antibody, or the antigen-binding fragments thereof.

3. The method of claim 2, further comprising determining the relative levels of at least two of human IgA, IgM, and IgG bound to the SARS-CoV-2 proteins or fragments thereof, thereby determining the immune response of the subject to a SARS-CoV-2 infection.

4. The method of claim 1, wherein the HRP substrate is 3,3,5,5′-tetramethylbenzidine.

5. The method of claim 1, wherein each of the wells is of a single multi-well plate.

6. The method of claim 1, wherein wells receiving the anti-human Ig antibody are of different multi-well plates.

7. The method of claim 1, wherein the method is a high-throughput assay.

8. The method of claim 1, wherein the bound SARS-CoV-2 comprises the SARS-CoV-2 spike protein from amino acid positions Arg319 to Phe541 (SEQ ID NO: 2) of the receptor-binding domain (RBD).

9. A kit comprising:
(i) a vessel or vessels containing at least one of a biotinylated anti-human IgA antibody, a biotinylated anti-human IgM antibody, and a biotinylated anti-human IgG antibody, or antigen-binding fragments thereof;
(ii) at least one multi-well plate comprising a plurality of wells, wherein the wells are coated with a mixture of a SARS-CoV-2 spike protein, or fragments thereof, and a SARS-CoV-2 nucleocapsid protein, or fragments thereof;
(iii) a vessel containing a horse radish peroxidase-streptavidin conjugate; and
(iv) instructions for the use of the reagents of the kit in the method of claim 1 for the detection of at least one of aSARS-CoV-2-specific IgA, IgM or IgG antibody.

10. The kit of claim 9, wherein the protein of SARS-CoV-2 is a SARS-CoV-2 spike protein.

11. The kit of claim 9, wherein the protein of SARS-CoV-2 comprises the SARS-CoV-2 spike protein from amino acid positions Arg319 to Phe541 (SEQ ID NO: 2) of the receptor-binding domain (RBD).
